(19) United States
(12) Patent Application Publication
(10) Pub. No.: US 2016/0202271 A1
(43) Pub. Date: Jul. 14, 2016

(54) TEST PAPER FOR DETECTION OF DIABETIC NEPHROPATHY

(71) Applicant: SHENZHEN LEADMED MEDICAL TECHNOLOGY COMPANY LIMITED, Shenzhen, Guangdong (CN)

(72) Inventors: Peng Yue, Shenzhen (CN); Jinyuan Zhou, Shenzhen (CN); Weiwei Sheng, Shenzhen (CN)

(21) Appl. No.: 14/655,748

(22) PCT Filed: Aug. 20, 2014
(86) PCT No.: PCT/CN2014/084800

§ 371 Date: Jun. 26, 2015

(30) Foreign Application Priority Data
Jul. 3, 2014 (CN) 201410317187.8

(51) Int. Cl. G01N 33/68; G01N 21/78

(52) U.S. Cl. CPC G01N 33/6893; G01N 21/78; G01N 2800/347; G01N 2800/042; G01N 2333/76; G01N 2333/4713; G01N 2021/7763

(57) ABSTRACT

Disclosed is a test paper for detection of diabetic nephropathy, which includes a water-absorbing filter paper, a glass fiber membrane and a NC membrane, all of which are stacked successively from top to bottom. The test paper has a T1 testing region, a T2 testing region and a T3 reference region arranged along a transverse direction; the NC membrane is coated by urinary microalbumin antibodies in the T1 testing region, is coated by urinary haptoglobin antibodies in the T2 testing region and is coated by mouse anti-human IgG antibodies in the T3 reference region; and the glass fiber membrane is coated by urinary microalbumin labeled with colloidal gold in the T1 testing region, is coated by urinary haptoglobin labeled with colloidal gold in the T2 testing region and is coated by mouse anti-human IgG albumen labeled with colloidal gold in the T3 reference region.

### FIELD OF THE INVENTION

The present invention relates to medicinal in-vitro diagnostic technology, in particular, it concerns a test paper for detection of diabetic nephropathy.

### BACKGROUND OF THE INVENTION

Diabetic nephropathy is a metabolic disease characterized by hyperglycemia. Hyperglycemia is caused by one or both of the defect of insulin secretion and the damage of biological action thereof. Long-standing hyperglycemia caused by diabetes will cause dysfunction and chronic damages of all kinds of tissues, especially eye, kidney, heart, blood vessel and nerve, all of which are called diabetic complications. Among the diabetic complications, chronic renal failure or uremia caused by prolonged hypertension and uncontrolled hyperglycaemia is a main cause of death of the advanced diabetics. Thus, it is very important to achieve early diagnosis of diabetic nephropathy for more than hundred million Chinese diabetics.

Traditional diagnostic method of early diabetic nephropathy has some disadvantages in all respects. For example, the traditional diagnostic method according to blood creatinine has a missed diagnosis rate reaching 62%; the diagnostic method according to urinary albumin suffers from the impact of many drugs thereby having a very high false positive rate and false negative rate; the traditional diagnostic method according to urinary microalbumin can serves as diagnosis standard of most of nephropathy but it is not suitable for the diabetic nephropathy caused by type II diabetes and its missed diagnosis rate reaches 50-60%; and several top international experimental results show that the urinary haptoglobin can be used to make a detection of diabetic nephropathy caused by type II diabetes by calculating the concentration of urine creatinine. However, because it needs to calculate the concentration of urine creatinine, it is inconvenient to operate and it is hard to be widely applied on clinical medicine for rapid and accurate diagnosis of diabetic nephropathy. At present, the “accurate diagnosis of diabetic nephropathy” is mainly achieved by reference to the creatinine clearance which is considered as a “gold standard” for diagnosis of the kidney disease and diabetic nephropathy. Such an index can reflect the glomerular filtration rate accurately, thereby the function of the kidney can be determined directly. However, due to the complicated diagnosis based on this index, it is rarely applied on clinical diagnosis. It not only needs complicated calculation formula, but also need to measuring the urinary flow (ml/h), the concentration of blood creatinine and the concentration of urine creatinine, thus the “gold standard” is mainly applied on medical research. Researches show that urinary haptoglobin is a new target of diabetic nephropathy and this target can be used to achieve an early detection of type II diabetes by calculating the concentration of urine creatinine. However, these researches are too academic and the diagnosis standard is not easy to operate, thus it is hard to be applied on clinical medicine for rapid and accurate diagnosis of diabetic nephropathy.

Test paper for detecting diabetic nephropathy is provided as a mean to test diabetic nephropathy, and it has the characteristics of rapid, simple and convenient and low cost. The whole test process takes only 3-10 minutes without any other apparatus. It is easy to operate without professionals; it is convenient to carry and can be used anytime and anywhere; and by means of the test papers, the samples can be detected in batch or individually. However, by means of colloidal gold immunochromatographic assay, the existing test paper can be used only for detecting the type I diabetes mellitus and type II diabetes mellitus, and due to its low correct rate of detection, its detecting result cannot be used as accurate diagnostic basis.

### SUMMARY OF THE INVENTION

For diabetic nephropathy, if the urinary microalbumin appears unusual, the urinary microalbumin in urine will increase; and on the contrary, if the urinary haptoglobin appears unusual, the urinary haptoglobin in urine will reduce. However, in many cases, one of the above indexes may be shown to be normal even for the patients with diabetic nephropathy, thereby causing a missed diagnosis. To solve the above problem, there are provided a test paper for rapid and accurate detection of diabetic nephropathy, which can be achieved by following solutions:

A test paper for detection of diabetic nephropathy includes a water-absorbing filter paper, a glass fiber membrane and a NC membrane, all of which are stacked successively from top to bottom. The test paper has a T1 testing region, a T2 testing region and a T3 reference region arranged along a transverse direction; the NC membrane is coated by urinary microalbumin antibodies in the T1 testing region, is coated by urinary haptoglobin antibodies in the T2 testing region and is coated by mouse anti-human IgG antibodies in the T3 reference region; and the glass fiber membrane is coated by urinary microalbumin labeled with colloidal gold in the T1 testing region, is coated by urinary haptoglobin labeled with colloidal gold in the T2 testing region and is coated by mouse anti-human IgG albumen labeled with colloidal gold in the T3 reference region.

The preparation method of the urinary microalbumin labeled with colloidal gold includes the steps of:

Taking 100 ml of 0.01% HAuCl₄ water solution, heating it to boiling, adding 2.5 ml of 1% trisodium citrate water solution rapidly and keeping it boiling until the solution becomes salmon pink, thereby obtaining colloidal gold solution;

Using urinary haptoglobin to dialysis low ionic strength water, removing tiny particles from the protein solution by microfiltration membrane or ultracentrifugation so as to get urinary microalbumin stock solution; taking the urinary microalbumin stock solution to be diluted to a series of diluents of 15 ug/ml, 20 ug/ml, 25 ug/ml, 30 ug/ml, 35 ug/ml, 40 ug/ml, 45 ug/ml, 50 ug/ml, respectively, taking 0.1 ml of each of the diluents to be added to 1 ml of the colloidal gold solution, respectively, providing a control group without urinary microalbumin, adding 0.1 ml of 10% NaCl solution after 5 min, mixing the solution thoroughly and letting it stand for 2 hours, and choosing the urinary microalbumin solution, which is capable of making the colloidal gold be most stable, to be added 10% colloidal gold solution so that the urinary microalbumin labeled with colloidal gold is achieved.

The preparation method of the urinary haptoglobin labeled with colloidal gold includes the steps of:

Taking 100 ml of 0.01% HAuCl₄ water solution, heating it to boiling, adding 1 ml of 1% trisodium citrate water solution rapidly and keeping it boiling until the solution becomes salmon pink, thereby obtaining colloidal gold solution;

Using urinary haptoglobin to dialysis low ionic strength water, removing tiny particles from the protein solution by microfiltration membrane or ultracentrifugation so as to get urinary haptoglobin stock solution; taking the urinary haptoglobin stock solution to be diluted to a series of diluents of 10 ug/ml, 15 ug/ml, 20 ug/ml, 25 ug/ml, 30 ug/ml, respectively, taking 0.1 ml of each of the diluents to be added to 1 ml of the colloidal gold solution, respectively, providing a control group without urinary haptoglobin, adding 0.1 ml of 10% NaCl solution after 5 min, mixing the solution thoroughly and letting it stand for 2 hours, and choosing the urinary haptoglobin solution, which is capable of making the colloidal gold be most stable, to be added 10% colloidal gold solution so that the urinary haptoglobin labeled with colloidal gold is achieved.

The preparation method of the mouse anti-human IgG albumen labeled with colloidal gold includes the steps of:

Taking 100 ml of 0.01% HAuCl₄ water solution, heating it to boiling, adding 4 ml of 1% trisodium citrate water solution rapidly and keeping it boiling until the solution becomes salmon pink, thereby obtaining colloidal gold solution;

Using mouse anti-human IgG albumen to dialysis low ionic strength water, removing tiny particles from the protein solution by microfiltration membrane or ultracentrifugation so as to get mouse anti-human IgG albumen stock solution; taking the mouse anti-human IgG albumen stock solution to be diluted to a series of diluents of 15 ug/ml, 25 ug/ml, 35 ug/ml, 45 ug/ml, 55 ug/ml, respectively, taking 0.1 ml of each of the diluents to be added to 1 ml of the colloidal gold solution, respectively, providing a control group without mouse anti-human IgG albumen, adding 0.1 ml of 10% NaCl solution after 5 min, mixing the solution thoroughly and letting it stand for 2 hours, and choosing the mouse anti-human IgG albumen solution, which is capable of making the colloidal gold be most stable, to be added 10% colloidal gold solution so that the mouse anti-human IgG albumen labeled with colloidal gold is achieved.

A method of application of the test paper for detection of diabetic nephropathy includes following steps: dropping urine onto the water-absorbing filter paper of one end of the test paper, by means of capillary action, the urine swimming to the T1 testing region, the T2 testing region and the T3 reference region so that the color and width of the T1 testing region, the T2 testing region and the T3 reference region are changed; measuring color density of the T1 testing region, the T2 testing region and the T3 reference region so as to determine the mass of each testing region and then obtaining the mass content of the urinary microalbumin and the urinary haptoglobin in the urine tested and added onto the T1 testing region and the T2 testing region.

The present invention has beneficial effects as follows: the test paper uses urinary microalbumin and urinary haptoglobin as biological targets; and when carrying out urine test, both of the content of urinary microalbumin and that of urinary haptoglobin in the urine can be detected out quickly and accurately and then the content ratio of urinary microalbumin and urinary haptoglobin in the urine can serve as diagnostic basis. It is simple to operate, it is fast and accurate and the rate of missed diagnosis is low. It can be applied on clinical medicine for rapid and accurate diagnosis of diabetic nephropathy, thereby supplying scientific diagnostic basis for preventing exacerbations of diabetic nephropathy.

### BRIEF DESCRIPTION OF THE DRAWINGS

FIG. 1 is a cross section view of a test paper for detection of diabetic nephropathy according to an embodiment of the present invention.

### DETAILED DESCRIPTION OF ILLUSTRATED EMBODIMENTS

For understanding the technical content of the present invention more sufficiently, now combine specific embodiments to introduce and illustrate the technical solution of the present invention as follows.

### EMBODIMENT

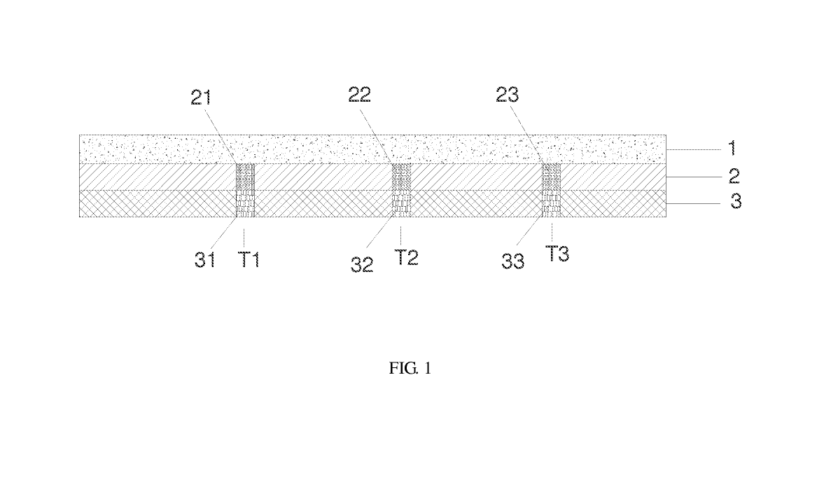

As shown in FIG. 1, the test paper for detection of diabetic nephropathy includes a water-absorbing filter paper 1, a glass fiber membrane 2 and a NC membrane 3, all of which are stacked successively from top to bottom. A T1 testing region, a T2 testing region and a T3 reference region are provided in the intermediate region of the test paper. The NC membrane 3 is coated by urinary microalbumin antibodies 31 in the T1 testing region, urinary haptoglobin antibodies 32 in the T2 testing region and mouse anti-human IgG antibodies 33 in the T3 reference region. The glass fiber membrane 2 is coated by urinary microalbumin 21 labeled with colloidal gold in the T1 testing region, urinary haptoglobin 22 labeled with colloidal gold in the T2 testing region and mouse anti-human IgG albumen 23 labeled with colloidal gold in the T3 reference region. The urinary microalbumin antibodies are various antibodies produced according to specific c-terminal structure of the urinary microalbumin. The urinary haptoglobin antibodies are various antibodies produced according to specific c-terminal structure of the urinary haptoglobin.

The preparation method of the urinary microalbumin labeled with colloidal gold includes following steps:

Take 100 ml of 0.01% HAuCl₄ water solution, heat it to boiling, add 2.5 ml of 1% trisodium citrate water solution rapidly and keep it boiling until the solution becomes salmon pink, thereby obtaining colloidal gold solution.

Use urinary microalbumin to dialysis low ionic strength water; remove tiny particles from the protein solution by microfiltration membrane or ultracentrifugation so as to get urinary microalbumin stock solution; take the urinary microalbumin stock solution to be diluted to a series of diluents of 15 ug/ml, 20 ug/ml, 25 ug/ml, 30 ug/ml, 35 ug/ml, 40 ug/ml, 45 ug/ml, 50 ug/ml, respectively, and take 0.1 ml of each of the diluents to be added to 1 ml of the colloidal gold solution, respectively; provide a control group without urinary microalbumin; add 0.1 ml of 10% NaCl solution after 5 min, mix the solution thoroughly and let it stand for 2 hours; and choose the urinary microalbumin solution, which is capable of making the colloidal gold be most stable, to be added 10% colloidal gold solution so that the urinary microalbumin labeled with colloidal gold is achieved.

The preparation method of the urinary haptoglobin labeled with colloidal gold includes following steps:

Take 100 ml of 0.01% HAuCl₄ water solution, heat it to boiling, add lml of 1% trisodium citrate water solution rapidly and keep it boiling until the solution becomes salmon pink, thereby obtaining colloidal gold solution.

Use urinary haptoglobin to dialysis low ionic strength water; remove tiny particles from the protein solution by microfiltration membrane or ultracentrifugation so as to get urinary haptoglobin stock solution; take the urinary haptoglobin stock solution to be diluted to a series of diluents of 10 ug/ml, 15 ug/ml, 20 ug/ml, 25 ug/ml, 30 ug/ml, respectively, and take 0.1 ml of each of the diluents to be added to 1 ml of the colloidal gold solution, respectively; provide a control group without urinary haptoglobin; add 0.1 ml of 10% NaCl solution after 5 min, mix the solution thoroughly and let it stand for 2 hours; and choose the urinary haptoglobin solution, which is capable of making the colloidal gold be most stable, to be added 10% colloidal gold solution so that the urinary haptoglobin labeled with colloidal gold is achieved.

The preparation method of the mouse anti-human IgG albumen labeled with colloidal gold includes following steps:

Take 100 ml of 0.01% HAuCl₄ water solution, heat it to boiling, add 4 ml of 1% trisodium citrate water solution rapidly and keep it boiling until the solution becomes salmon pink, thereby obtaining colloidal gold solution.

Use mouse anti-human IgG albumen to dialysis low ionic strength water; remove tiny particles from the protein solution by microfiltration membrane or ultracentrifugation so as to get mouse anti-human IgG albumen stock solution; take the mouse anti-human IgG albumen stock solution to be diluted to a series of diluents of 15 ug/ml, 25 ug/ml, 35 ug/ml, 45 ug/ml, 55 ug/ml, respectively, and take 0.1 ml of each of the diluents to be added to 1 ml of the colloidal gold solution, respectively; provide a control group without mouse anti-human IgG albumen; add 0.1 ml of 10% NaCl solution after 5 min, mix the solution thoroughly and let it stand for 2 hours; and choose the mouse anti-human IgG albumen solution, which is capable of making the colloidal gold be most stable, to be added 10% colloidal gold solution so that the mouse anti-human IgG albumen labeled with colloidal gold is achieved.

The glass fiber membrane with combination of colloidal gold and albumen is prepared by the steps of: spraying the urinary microalbumin labeled with colloidal gold, the urinary haptoglobin labeled with colloidal gold and the mouse anti-human IgG albumen labeled with colloidal gold into the T1 testing region, the T2 testing region and the T3 reference region of the glass fiber membrane, respectively, by a spraying apparatus; and then drying it in vacuum at 37° C. for 2 hours.

The NC membrane coated by antibodies is prepared by the steps of:

Take the urinary microalbumin antibodies to be diluted by factors of 2, 4, 8 and 16, respectively, by using PBS. Take standard urine to react with each diluted antibodies solution, and then drop the antibodies solution, which is suitable for tigen-antibody reaction, onto the T1 testing region of the NC membrane via a 1 μl pipettor.

Take the urinary haptoglobin antibodies to be diluted by factors of 2, 4, 8 and 16, respectively, by using PBS. Take standard urine to react with each diluted antibodies solution, and then drop the antibodies solution, which is suitable for tigen-antibody reaction, onto the T2 testing region of the NC membrane via a 1 μl pipettor.

Take the mouse anti-human IgG antibodies to be diluted by factors of 2, 4, 8 and 16, respectively, by using PBS. Take standard urine to react with each diluted antibodies solution, and then drop the antibodies solution, which is suitable for tigen-antibody reaction, onto the T3 reference region of the NC membrane via a 1 μl pipettor.

Dry the NC membrane coated by antibodies at 37° C. for 1 hour.

Assemble the water-absorbing filter paper 1, the glass fiber membrane 2 and the NC membrane 3 into a test paper.

The method of application of the test paper for detection of diabetic nephropathy includes following steps: dropping urine onto the water-absorbing filter paper of one end of the test paper, by means of capillary action, the urine swimming to the T1 testing region, the T2 testing region and the T3 reference region so that the color and width of the T1 testing region, the T2 testing region and the T3 reference region are changed; measuring color density of the T1 testing region, the T2 testing region and the T3 reference region so as to determine the mass of each testing region and then obtaining the mass content of the urinary microalbumin and the urinary haptoglobin in the urine tested and added onto the T1 testing region and the T2 testing region.

The test paper of the present invention uses urinary microalbumin and urinary haptoglobin as biological targets so that both of the content of urinary microalbumin and that of the urinary haptoglobin in the urine can be detected out quickly and accurately. By means of calculating the content ratio of the urinary microalbumin and the urinary haptoglobin in the urine, a diagnosis of diabetic nephropathy can be achieved, and the rate of missed diagnosis is low (about 1-2%). Due to the difference of changes of the two indexs in the diagnosis of diabetic nephropathy, the two indexs are combined to be one value by means of the above calculation formula, thereby improving the diagnosis sensibility of the value. Thus it is easy to be applied on clinical medicine. The judgement standard of the mass content ratio of the urinary microalbumin and the urinary haptoglobin in the urine is: normal range being less than or equal to 0.8; abnormal range being more than 0.8.

The test methods for making diagnoses for test subjects, which are patients with diabetic nephropathy, by a diagnostic method of using the test paper of the present invention, a diagnostic method of measuring urinary albumin, a diagnostic method of measuring urinary microalbumin via test paper of colloidal gold and a diagnostic method of measuring urinary haptoglobin via test paper of colloidal gold, will be described as follows:

(1) Feed adult wistar big rats of 150˜200 g for one month in high fat way and then keep feeding the big rats in high fat way, inject streptozotocin(STZ) in abdominal cavity of the big rats for two weeks, and measure (serum creatinine/urine creatinine)*urine flow of the adult wistar big rats. Wherein the urine flow is expressed in milliliter per hour, and is measured by recording interval time of two urine generation, collecting the urine after the interval time and then calculating the urine flow according to a formula of urine volume/time. The serum creatinine and the urine creatinine can be measured by biochemistry kit.

Choose 300 big rats of abnormal index, measure the urinary protein in the urine by biochemical method, measure the urinary microalbumin and the urinary haptoglobin by test paper for detection of diabetic nephropathy of the present invention, and then calculate the ratio of the urinary microalbumin and the haptoglobin, and finally determine the ratios of diabetic nephropathy diagnosed by various methods among the big rats.

(2) Feed db/db small rats of 20˜30 g for two weeks and measure (serum creatinine/urine creatinine)*urine flow of the small rats. Wherein the urine flow is expressed in milliliter per hour, and is measured by recording interval time of two urine generation, collecting the urine after the interval time and then calculating the urine flow according to a formula of urine volume/time. The serum creatinine and the urine creatinine can be measured by biochemistry kit.

Choose 300 small rats of abnormal index, measure the urinary protein in the urine by biochemical method, measure the urinary microalbumin and the urinary haptoglobin by test paper for detection of diabetic nephropathy of the present invention, and then calculate the ratio of the urinary microalbumin and the haptoglobin, and finally determine the ratios of diabetic nephropathy diagnosed by various methods among the small rats.

(3) Choose type I diabetics and measure (serum creatinine/urine creatinine)*urine flow of the type I diabetics. Wherein the urine flow is expressed in milliliter per hour, and is measured by recording interval time of two urine generation, collecting the urine after the interval time and then calculating the urine flow according to a formula of urine volume/time. The serum creatinine and the urine creatinine can be measured by biochemistry kit.

Choose 220 type I diabetics of abnormal index, measure the urinary protein in the urine by biochemical method, measure the urinary microalbumin and the urinary haptoglobin by test paper for detection of diabetic nephropathy of the present invention, and then calculate the ratio of the urinary microalbumin and the haptoglobin, and finally determine the ratios of diabetic nephropathy diagnosed by various methods among the type I diabetics.

(4) Choose type II diabetics and measure (serum creatinine/urine creatinine)*urine flow of the type II diabetics. Wherein the urine flow is expressed in milliliter per hour, and is measured by recording interval time of two urine generation, collecting the urine after the interval time and then calculating the urine flow according to a formula of urine volume/time. The serum creatinine and the urine creatinine can be measured by biochemistry kit.

Choose 235 type II diabetics of abnormal index, measure the urinary protein in the urine by biochemical method, measure the urinary microalbumin and the urinary haptoglobin by test paper for detection of diabetic nephropathy of the present invention, and then calculate the ratio of the urinary microalbumin and the haptoglobin, and finally determine the ratios of diabetic nephropathy diagnosed by various methods among the type II diabetics.

The statistical results of the ratios of diabetic nephropathy diagnosed by various methods are shown in following table 1.

TABLE 1

| | | Small | Type I | Type II |
|---|---|---|---|---|
| Diagnostic method | Big rats | rats | diabetics | diabetics |
| Measuring urinary | 45.3% | 35.3% | 25.5% | 28.1% |
| protein | | | | |
| Measuring urinary | 35.3% | 72.3% | 97.3% | 30.2% |
| microalbumin | | | | |
| Measuring urinary | 81.3% | 72.3% | 82.3% | 80.4% |
| haptoglobin | | | | |
| Measuring urinary | 98.7% | 99% | 99.1% | 98.7% |
| microalbumin/urinary | | | | |
| haptoglobin | | | | |

Because all of the above samples are compared with each other by determining (serum creatinine/urine creatinine)*urine flow and such an index is an acknowledged gold standard for judgment of diabetic nephropathy in experimental research, all of the testing bodies in the testing programs, of which the index of (serum creatinine/urine creatinine)*urine flow is abnormal, are diabetic patients. In view of the above results, the diagnostic method by measuring microalbumin/urinary haptoglobin is most accurate and effective for clinical identification of diabetic nephropathy.

While the invention has been described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention.

What is claimed is:

1. A test paper for detection of diabetic nephropathy, comprising a water-absorbing filter paper, a glass fiber membrane and a NC membrane, all of which are stacked successively from top to bottom; the test paper having a T1 testing region, a T2 testing region and a T3 reference region arranged along a transverse direction; the NC membrane being coated by urinary microalbumin antibodies in the T1 testing region, being coated by urinary haptoglobin antibodies in the T2 testing region and being coated by mouse anti-human IgG antibodies in the T3 reference region; the glass fiber membrane being coated by urinary microalbumin labeled with colloidal gold in the T1 testing region, being coated by urinary haptoglobin labeled with colloidal gold in the T2 testing region and being coated by mouse anti-human IgG albumen labeled with colloidal gold in the T3 reference region.

2. The test paper for detection of diabetic nephropathy according to claim 1, wherein the urinary microalbumin labeled with colloidal gold is prepared by:
taking 100 ml of 0.01% HAuCl₄ water solution, heating it to boiling, adding 2.5 ml of 1% trisodium citrate water solution rapidly and keeping it boiling until the solution becomes salmon pink, thereby obtaining colloidal gold solution;
using urinary haptoglobin to dialysis low ionic strength water, removing tiny particles from the protein solution by microfiltration membrane or ultracentrifugation so as to get urinary microalbumin stock solution; taking the urinary microalbumin stock solution to be diluted to a series of diluents of 15 ug/ml, 20 ug/ml, 25 ug/ml, 30 ug/ml, 35 ug/ml, 40 ug/ml, 45 ug/ml, 50 ug/ml, respectively, taking 0.1 ml of each of the diluents to be added to 1 ml of the colloidal gold solution, respectively, providing a control group without urinary microalbumin, adding 0.1 ml of 10% NaCl solution after 5 min, mixing the solution thoroughly and letting it stand for 2 hours, and choosing the urinary microalbumin solution, which is capable of making the colloidal gold be most stable, to be added 10% colloidal gold solution so that the urinary microalbumin labeled with colloidal gold is achieved.

3. The test paper for detection of diabetic nephropathy according to claim 1, wherein the urinary haptoglobin labeled with colloidal gold is prepared by:
taking 100 ml of 0.01% HAuCl₄ water solution, heating it to boiling, adding 1 ml of 1% trisodium citrate water solution rapidly and keeping it boiling until the solution becomes salmon pink, thereby obtaining colloidal gold solution;
using urinary haptoglobin to dialysis low ionic strength water, removing tiny particles from the protein solution by microfiltration membrane or ultracentrifugation so as to get urinary haptoglobin stock solution; taking the urinary haptoglobin stock solution to be diluted to a series of diluents of 10 ug/ml, 15 ug/ml, 20 ug/ml, 25 ug/ml, 30 ug/ml, respectively, taking 0.1 ml of each of the diluents to be added to 1 ml of the colloidal gold solution, respectively, providing a control group without urinary haptoglobin, adding 0.1 ml of 10% NaCl solution after 5 min, mixing the solution thoroughly and letting it stand for 2 hours, and choosing the urinary haptoglobin solution, which is capable of making the colloidal gold be most stable, to be added 10% colloidal gold solution so that the urinary haptoglobin labeled with colloidal gold is achieved.

4. The test paper for detection of diabetic nephropathy according to claim 1, wherein the mouse anti-human IgG albumen labeled with colloidal gold is prepared by:
taking 100 ml of 0.01% HAuCl₄ water solution, heating it to boiling, adding 4 ml of 1% trisodium citrate water solution rapidly and keeping it boiling until the solution becomes salmon pink, thereby obtaining colloidal gold solution;
using mouse anti-human IgG albumen to dialysis low ionic strength water, removing tiny particles from the protein solution by microfiltration membrane or ultracentrifugation so as to get mouse anti-human IgG albumen stock solution; taking the mouse anti-human IgG albumen stock solution to be diluted to a series of diluents of 15 ug/ml, 25 ug/ml, 35 ug/ml, 45 ug/ml, 55 ug/ml, respectively, taking 0.1 ml of each of the diluents to be added to 1 ml of the colloidal gold solution, respectively, providing a control group without mouse anti-human IgG albumen, adding 0.1 ml of 10% NaCl solution after 5 min, mixing the solution thoroughly and letting it stand for 2 hours, and choosing the mouse anti-human IgG albumen solution, which is capable of making the colloidal gold be most stable, to be added 10% colloidal gold solution so that the mouse anti-human IgG albumen labeled with colloidal gold is achieved.

5. A method of application of the test paper for detection of diabetic nephropathy according to claim 1, comprising following steps: dropping urine onto the water-absorbing filter paper of one end of the test paper, by means of capillary action, the urine swimming to the T1 testing region, the T2 testing region and the T3 reference region so that the color and width of the T1 testing region, the T2 testing region and the T3 reference region are changed; measuring color density of the T1 testing region, the T2 testing region and the T3 reference region so as to determine the mass of each testing region and then obtaining the mass content of the urinary microalbumin and the urinary haptoglobin in the urine tested and added onto the T1 testing region and the T2 testing region.
